(19) United States
(12) Patent Application Publication
(10) Pub. No.: US 2011/0166436 A1
(43) Pub. Date: Jul. 7, 2011

(54) System and Method For Non-Contrast MR Angiography Using Steady-State Image Acquisition

(76) Inventor: Robert R. Edelman, Highland Park, IL (US)

(21) Appl. No.: 12/651,801

(22) Filed: Jan. 4, 2010

(51) Int. Cl. A61B 5/055; G06K 9/00

(52) U.S. Cl. 600/410; 382/130

(57) ABSTRACT

A system and method is provided to quickly acquire and produce an MR angiogram without the use of a contrast agent. In quick succession, two MR image data sets of the vasculature of interest are acquired using a steady-state free precession (SSFP) pulse sequence. The SSFP pulse sequence gradient pulses differ for each image acquisition in that gradient pulses are balanced, or first moment nulled, for one acquisition, but not the other. Magnitude images are reconstructed from the two acquired image data sets and the magnitude images are subtracted to produce the MR angiogram. Contrast is provided by spin motion without the use of contrast agents and without the time consuming addition of motion encoding gradients or preparatory pulse sequences.

### CROSS-REFERENCE TO RELATED APPLICATIONS

This application claims the benefit of U.S. Provisional Patent Application Ser. No. 61/142,182, filed on Jan. 2, 2009, and entitled “SYSTEM AND METHOD FOR NON-CONTRAST AGENT MR ANGIOGRAPHY,” which is incorporated herein by reference.

### FIELD OF THE INVENTION

The invention relates to a system and method for performing magnetic resonance angiography (MRA) and, more particularly, to a system and method for performing MRA without the need of a contrast agent.

### BACKGROUND OF THE INVENTION

When a substance such as human tissue is subjected to a uniform magnetic field (polarizing field B₀), the individual magnetic moments of the nuclear spins in the tissue attempt to align with this polarizing field, but precess about it in random order at their characteristic Larmor frequency. Usually the nuclear spins are comprised of hydrogen atoms, but other NMR active nuclei are occasionally used. A net magnetic moment M_{Z} is produced in the direction of the polarizing field, but the randomly oriented magnetic components in the perpendicular, or transverse, plane (x-y plane) cancel one another. If, however, the substance, or tissue, is subjected to a magnetic field (excitation field B₁; also referred to as the radiofrequency (RF) field) which is in the x-y plane and which is near the Larmor frequency, the net aligned moment, M_{z}, may be rotated, or “tipped” into the x-y plane to produce a net transverse magnetic moment Mₜ, which is rotating, or spinning, in the x-y plane at the Larmor frequency. The practical value of this phenomenon resides in the signal which is emitted by the excited spins after the excitation field B₁ is terminated. There are a wide variety of measurement sequences in which this nuclear magnetic resonance (“NMR”) phenomenon is exploited.

When utilizing these signals to produce images, magnetic field gradients (Gₓ, G_{y}, and G_{z}) are employed. Typically, the region to be imaged experiences a sequence of measurement cycles in which these gradients vary according to the particular localization method being used. The emitted MR signals are detected using a receiver coil. The MRI signals are then digitized and processed to reconstruct the image using one of many well-known reconstruction techniques.

The ability to depict anatomy and pathology using MRI is dependent on the contrast, or difference in signal intensity between the target and background tissue. In order to maximize contrast, it is necessary to suppress the signal intensities of the background tissues. For instance, small blood vessels are much better depicted by the technique of MRA when the signal intensities of fat and muscle (background tissues) are minimized.

Magnetic resonance angiography (MRA) uses the NMR phenomenon to produce images of the human vasculature. There are three main categories of techniques for achieving the desired contrast for the purpose of MR angiography. The first general category is typically referred to as contrast enhanced (CE) MRA. The second general category is time-of-flight (TOF) MRA. The third general category is phase contrast (PC) MRA.

To perform CE MRA, a contrast agent, such as gadolinium, is injected into the patient prior to the magnetic resonance (MR) angiogram to enhance the diagnostic capability of the MR angiogram. Contrast enhanced MRA attempts to acquire the central k-space views at the moment the bolus of contrast agent is flowing through the vasculature being imaged. Collection of the central lines of k-space during peak arterial enhancement is important to the success of a CE MRA exam. If the central lines of k-space are acquired prior to the arrival of contrast, severe image artifacts can limit the diagnostic information in the image. Alternatively, arterial images acquired after the passage of the peak arterial contrast are sometimes obscured by the enhancement of veins.

While CE MRA is a highly effective means for noninvasively evaluating suspected vascular disease, the technique suffers from several additional drawbacks. First, the contrast agent that must be administered to enhance the blood vessel carries a significant financial cost. Second, contrast agents such as gadolinium have recently been shown to be causative of an often catastrophic disorder called nephrogenic systemic fibrosis (NSF). Third, CE MRA does not provide hemodynamic information, so that it is not always feasible to determine if a stenosis is hemodynamically significant. Fourth, the signal-to-noise ratio (SNR) and, therefore, spatial resolution is limited by the need to acquire data quickly during the first pass of contrast agent through a target vessel.

The 3D time-of-flight (TOF) techniques were introduced in the 1980s and they have changed little over the last decade. The 3D TOF MRA techniques commonly used for cranial examinations and have not been replaced despite recent advances in time-resolved contrast-enhanced 3D MRA. An alternative technique known as pulsed arterial spin labeling (PASL) was first applied to image intracranial circulation years ago; however, image quality never approached that of 3D TOF and the method has had little clinical utility. Moreover, electrocardiographic (ECG) gating was required. The use of TOF MRA is generally limited to imaging of intracranial circulation, however, because of sensitivity to patient motion and flow artifacts.

Finally, phase contrast MRA is largely reserved for the measurement of flow velocities and imaging of veins. It requires a longer scan time and the operator must set a velocity-encoding sensitivity, which varies unpredictably depending on a variety of clinical factors.

More recent MRA methods have also been proposed. The signal targeting with alternating radiofrequency (STAR) technique, developed by Edelman et al. more than a decade ago, involves the application of an inversion B₁ pulse to spins outside of a selected region to be imaged, and not to the imaged region itself. The technique relies on the subtraction of two images sets in which background tissues have been exposed to precisely the same RF pulses. The STAR technique is ideally suited for imaging blood vessels containing fast blood flow, such as arteries, and is not well suited for imaging of veins containing slow blood flow.

The flow-sensitive alternating inversion recovery (FAIR) technique, along with the related FAIR with extra radiofrequency pulse (FAIRER) technique, applies a spatially non-selective inversion in one acquisition, and a spatially selective inversion to a region in the other acquisition. As in the case of STAR, it relies on the subtraction of two images sets in which background tissues have been exposed to precisely the same RF pulses. The method is primarily used for functional imaging of the brain and has not been used for MR angiography. It relies on inflow of spins into the selected region and is not suitable for imaging of veins. Moreover, it is highly sensitive to magnetization transfer effects that can result in imperfect image subtraction.

A new MRA method known as STARFIRE has been proposed by Robert Edelman and is disclosed in pending U.S. patent application Ser. No. 12/257,066 entitled “System and Method for Non-Contrast Agent MR Angiography.” This method includes making two data acquisitions: one with a preparatory pulse sequence in which blood is suppressed, fat is recovered and other tissues are reduced; and a second in which the signals from blood is recovered, fat is substantially recovered and other tissues are reduced. Subtracting the two acquired image data sets results in an angiogram in which blood vessels have enhanced brightness. However fluids and edema also appear bright in a STARFIRE angiogram and this can obscure the view of blood vessel details in some clinical situations.

Therefore, it would be desirable to have a system and method for MRA that does not suffer from the drawbacks of each of the methods described above.

### SUMMARY OF THE INVENTION

The present invention provides a method for producing an angiogram with a magnetic resonance imaging (MRI) system without the need for administering a contrast agent and does not suffer from other drawbacks of the methods described above. More specifically, the present method includes acquiring in quick succession two MR image data sets of the vasculature of interest using a steady-state free precession (SSFP) pulse sequence. The SSFP pulse sequence gradient pulses differ for each image acquisition in that gradient pulses are balanced, or first moment nulled, for one acquisition, but not the other. Magnitude images are reconstructed from the two acquired image data sets and the magnitude images are subtracted to produce the MR angiogram.

The present invention also provides a non-contrast agent MR angiogram which substantially eliminates MR signals from edematous tissues. Such signals appear bright in other methods, such as STARFIRE, and they can interfere with the visualization of blood vessels in the legs or in the vicinity of tumors or inflamed tissues.

### BRIEF DESCRIPTION OF THE DRAWINGS

FIG. 1 is a block diagram of an MRI system for use with the present invention;

FIG. 2 is a schematic representation of a transceiver system for use with the MRI system of FIG. 1;

FIG. 3 is a diagram illustrating two SSFP pulse sequences performed by the MRI system of FIG. 1 in accordance with the present invention; and

FIG. 4 is a flow chart of the steps performed in accordance with one exemplary implementation of the present invention.

### DETAILED DESCRIPTION OF THE INVENTION

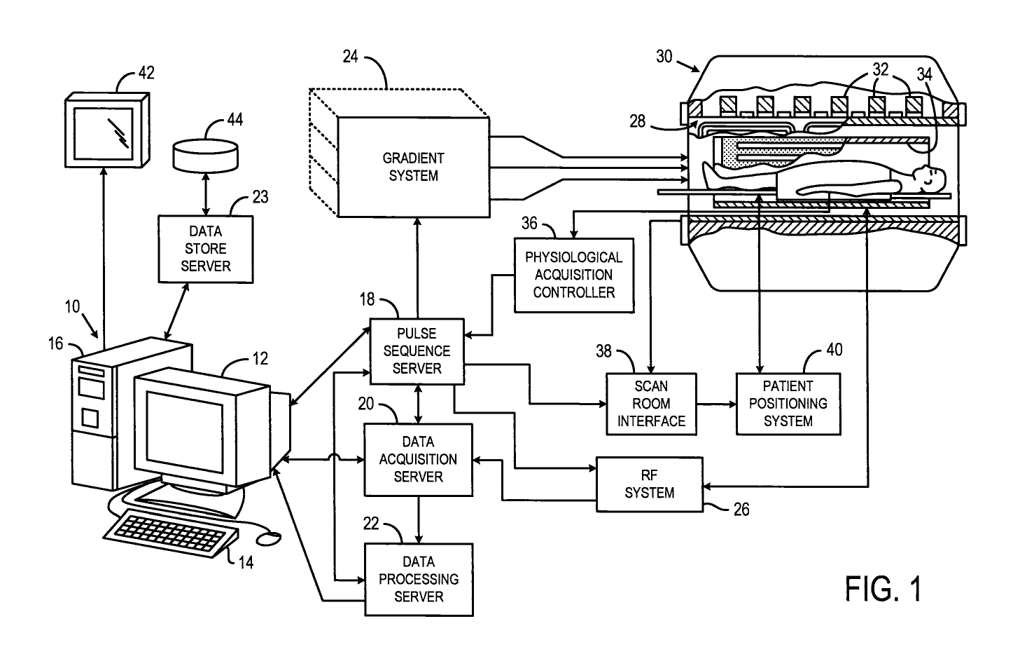

Referring particularly to FIG. 1, the invention is employed in an MRI system. The MRI system includes a workstation 10 having a display 12 and a keyboard 14. The workstation 10 includes a processor 16 that is a commercially available programmable machine running a commercially available operating system. The workstation 10 provides the operator interface that enables scan prescriptions to be entered into the MRI system.

The workstation 10 is coupled to, for example, four servers, including a pulse sequence server 18, a data acquisition server 20, a data processing server 22, and a data store server 23. In one configuration, the data store server 23 is performed by the workstation processor 16 and associated disc drive interface circuitry and the remaining three servers 18, 20, 22 are performed by separate processors mounted in a single enclosure and interconnected using a backplane bus. The pulse sequence server 18 employs a commercially available microprocessor and a commercially available communication controller. The data acquisition server 20 and data processing server 22 both employ commercially available microprocessors and the data processing server 22 further includes one or more array processors based on commercially available processors.

The workstation 10 and each processor for the servers 18, 20, 22 are connected to a communications network. This network conveys data that is downloaded to the servers 18, 20, 22 from the workstation 10 and conveys data that is communicated between the servers 18, 20, 22 and between the workstation 10 and the servers 18, 20, 22. In addition, a high speed data link is typically provided between the data processing server 22 and the workstation 10 in order to convey image data to the data store server 23.

The pulse sequence server 18 functions in response to program elements downloaded from the workstation 10 to operate a gradient system 24 and an RF system 26. Gradient waveforms necessary to perform the prescribed scan are produced and applied to the gradient system 24 that excites gradient coils in an assembly 28 to produce the magnetic field gradients Gₓ, G_{y}, and G_{z} used for position encoding NMR signals. The gradient coil assembly 28 forms part of a magnet assembly 30, which includes a polarizing magnet 32 and a whole-body RF coil 34.

The RF excitation waveforms are applied to the RF coil 34 by the RF system 26 to perform the prescribed magnetic resonance pulse sequence. Responsive NMR signals detected by the RF coil 34 are received by the RF system 26, amplified, demodulated, filtered, and digitized under direction of commands produced by the pulse sequence server 18. The RF system 26 includes an RF transmitter for producing a wide variety of RF pulses used in MR pulse sequences. The RF transmitter is responsive to the scan prescription and direction from the pulse sequence server 18 to produce RF pulses of the desired frequency, phase, and pulse amplitude waveform. The generated RF pulses may be applied to the whole body RF coil 34 or to one or more local coils or coil arrays.

The RF system 26 also includes one or more RF receiver channels. Each RF receiver channel includes an RF amplifier that amplifies the NMR signal received by the coil to which it is connected and a quadrature detector that detects and digitizes the in-phase (I) and quadrature (Q) components of the received NMR signal. The magnitude of the received NMR signal may thus be determined at any sampled point by the square root of the sum of the squares of the I and Q components.

The pulse sequence server 18 also optionally receives patient data from a physiological acquisition controller 36. The controller 36 receives signals from a number of different sensors connected to the patient, such as ECG signals from electrodes or respiratory signals from a bellows. Such signals are typically used by the pulse sequence server 18 to synchronize, or “gate”, the performance of the scan with the subject's respiration or heart beat.

The pulse sequence server 18 also connects to a scan room interface circuit 38 that receives signals from various sensors associated with the condition of the patient and the magnet system. It is also through the scan room interface circuit 38 that a patient positioning system 40 receives commands to move the patient to desired positions during the scan.

It should be apparent that the pulse sequence server 18 performs real-time control of MRI system elements during a scan. As a result, it is necessary that its hardware elements be operated with program instructions that are executed in a timely manner by run-time programs. The description components for a scan prescription are downloaded from the workstation 10 in the form of objects. The pulse sequence server 18 contains programs that receive these objects and converts them to objects that are employed by the run-time programs.

The digitized NMR signal samples produced by the RF system 26 are received by the data acquisition server 20. The data acquisition server 20 operates in response to description components downloaded from the workstation 10 to receive the real-time NMR data and provide buffer storage such that no data is lost by data overrun. In some scans, the data acquisition server 20 does little more than pass the acquired NMR data to the data processor server 22. However, in scans that require information derived from acquired NMR data to control the further performance of the scan, the data acquisition server 20 is programmed to produce such information and convey it to the pulse sequence server 18. For example, during prescans NMR data is acquired and used to calibrate the pulse sequence performed by the pulse sequence server 18. Also, navigator signals may be acquired during a scan and used to adjust RF or gradient system operating parameters or to control the view order in which k-space is sampled. Furthermore, the data acquisition server 20 may be employed to process NMR signals used to detect the arrival of contrast agent in an MRA scan. In all these examples the data acquisition server 20 acquires NMR data and processes it in real-time to produce information that is used to control the scan.

The data processing server 22 receives NMR data from the data acquisition server 20 and processes it in accordance with description components downloaded from the workstation 10. Such processing may include, for example, Fourier transformation of raw k-space NMR data to produce two or three-dimensional images, the application of filters to a reconstructed image, the performance of a backprojection image reconstruction of acquired NMR data, the calculation of functional MR images, the calculation of motion or flow images, and the like.

Images reconstructed by the data processing server 22 are conveyed back to the workstation 10 where they are stored. Real-time images are stored in a data base memory cache (not shown) from which they may be output to operator display 12 or a display 42 that is located near the magnet assembly 30 for use by attending physicians. Batch mode images or selected real time images are stored in a host database on disc storage 44. When such images have been reconstructed and transferred to storage, the data processing server 22 notifies the data store server 23 on the workstation 10. The workstation 10 may be used by an operator to archive the images, produce films, or send the images via a network to other facilities.

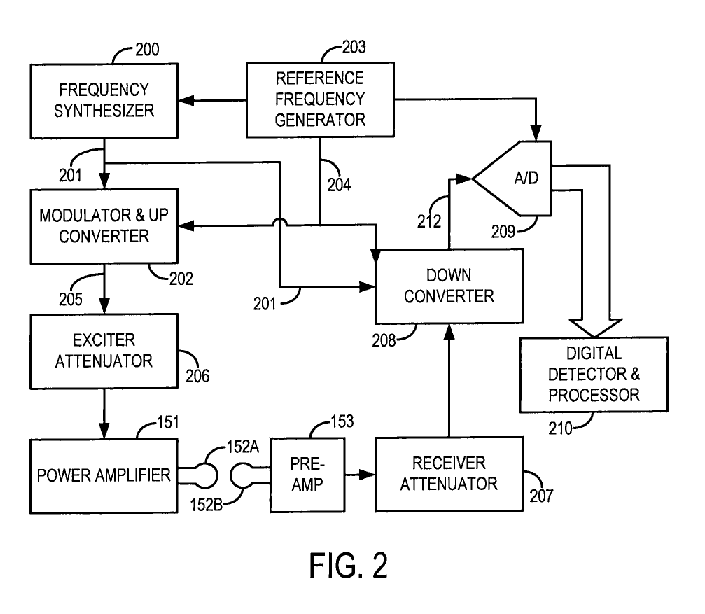

As shown in FIG. 1, the RF system 26 may be connected to the whole body RF coil 34, or as shown in FIG. 2, a transmitter section of the RF system 26 may connect to one RF coil 151A and its receiver section may connect to a separate RF receive coil 151B. Often, the transmitter section is connected to the whole body RF coil 34 and each receiver section is connected to a separate local coil 151B.

Referring particularly to FIG. 2, the RF system 26 includes a transmitter that produces a prescribed RF excitation field. The base, or carrier, frequency of this RF excitation field is produced under control of a frequency synthesizer 200 that receives a set of digital signals from the pulse sequence server 18. These digital signals indicate the frequency and phase of the RF carrier signal produced at an output 201. The RF carrier is applied to a modulator and up converter 202 where its amplitude is modulated in response to a signal R(t) also received from the pulse sequence server 18. The signal R(t) defines the envelope of the RF excitation pulse to be produced and is produced by sequentially reading out a series of stored digital values. These stored digital values may, be changed to enable any desired RF pulse envelope to be produced.

The magnitude of the RF excitation pulse produced at output 205 is attenuated by an exciter attenuator circuit 206 that receives a digital command from the pulse sequence server 18. The attenuated RF excitation pulses are applied to the power amplifier 151 that drives the RF coil 151A.

Referring still to FIG. 2, the signal produced by the subject is received by the receiver coil 152B and applied through a preamplifier 153 to the input of a receiver attenuator 207. The receiver attenuator 207 further amplifies the signal by an amount determined by a digital attenuation signal received from the pulse sequence server 18. The received signal is at or around the Larmor frequency, and this high frequency signal is down converted in a two step process by a down converter 208 that first mixes the NMR signal with the carrier signal on line 201 and then mixes the resulting difference signal with a reference signal on line 204. The down converted NMR signal is applied to the input of an analog-to-digital (A/D) converter 209 that samples and digitizes the analog signal and applies it to a digital detector and signal processor 210 to produce the I values and Q values corresponding to the received signal. As described above, the resulting stream of digitized I and Q values of the received signal are output to the data acquisition server 20 of FIG. 1. The reference signal, as well as the sampling signal applied to the A/D converter 209, is produced by a reference frequency generator 203.

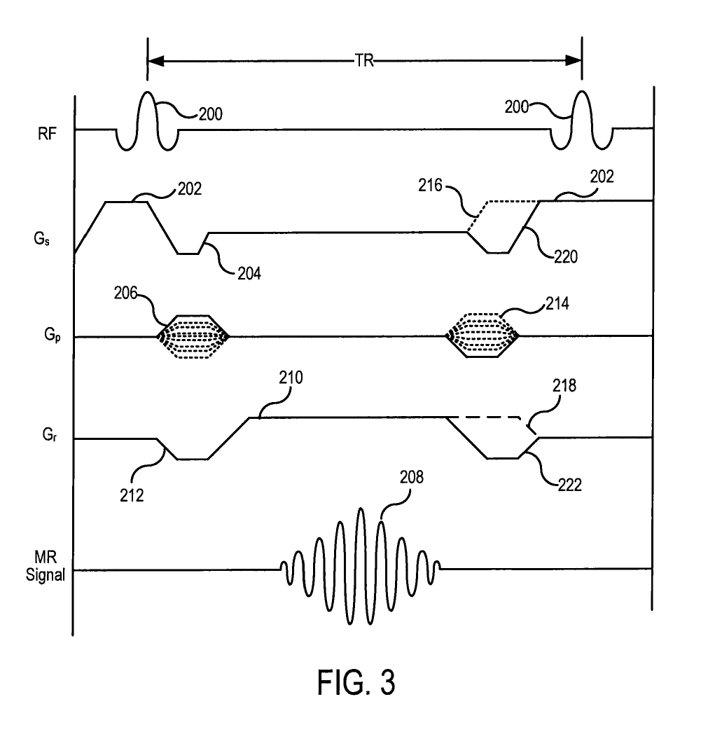

Referring particularly to FIG. 3, a pulse sequence diagram consistent with the present invention is provided. The pulse sequences employed with the present invention are, for example, a balanced SSFP pulse sequence, such as available on Siemens MR scanners as “TrueFISP,” and a unbalanced SSFP pulse sequence, such as available on the Siemens scanner as “FISP.” Both pulse sequences begin with a selective RF excitation pulse 200 played out in the presence of a slice encoding gradient pulse 202, followed by a slice select gradient rephrasing lobe 204. A phase encoding gradient pulse 206 is then applied to the resulting transverse magnetization and then an MR signal 208 is acquired in the presence of a readout gradient lobe 210. A readout gradient dephasing lobe 212 is played out prior to the readout gradient lobe 210 to produce the MR gradient echo signal 208 during the signal readout. The phase encoding is then rewound with a phase encoding rewinder gradient pulse 214. The pulse sequence is repeated many times during a scan and the phase encoding gradient pulse 206 is sequenced through a series of values to sample k-space in the prescribed manner. The rewinder gradient pulse 214 is sequenced through the same values, but it is always opposite in polarity to rephase spins in preparation for the next pulse sequence to follow. The SSFP pulse sequence is characterized by a very short duration (TR) and a flip-angle produced by its RF excitation pulse 200 that results in a steady-state longitudinal magnetization when the pulse sequences are played out in rapid succession.

The differences between the balanced and unbalanced SSFP pulse sequences are depicted by the dotted line 216 on the slice select gradient and the dotted line 218 on the readout gradient. The dotted lines 216 and 218 illustrate the gradients played out by the unbalanced SSFP pulse sequence and line segments 220 and 222 show the corresponding gradients played out by the balanced SSFP pulse sequence. The balanced slice select and readout gradients are shaped to have a nulled first moment that rephases the signal from moving spins from one pulse sequence to the next such that the MR signals acquired from moving spins remain high or bright during the scan. The same is not true of the unbalanced SSFP pulse sequence and the slice select and readout gradients dephase the signals from moving spins as the SSFP pulse sequences are played out. As a result, the MR signals from moving spins are suppressed and appear dark.

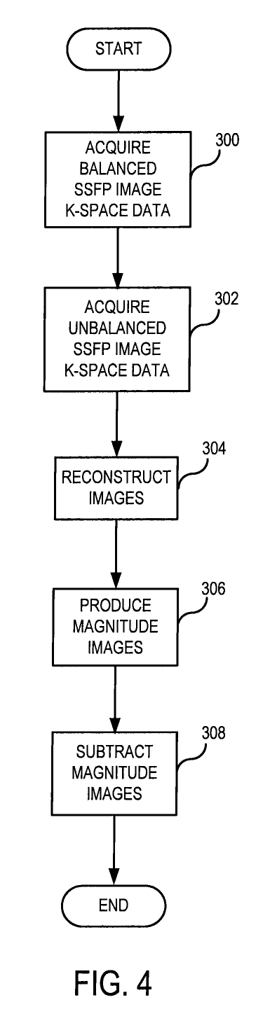

The balanced and unbalanced SSFP pulse sequences are employed to acquire images of the vasculature of interest. Referring particularly to FIG. 4., a flow chart setting forth the steps of an imaging process in accordance with the present invention is provided. In particular, after placing the subject in the bore of the MRI system and properly aligning the vasculature of interest in the field of view, a k-space image data set is acquired as indicated at process block 300 using the balanced SSFP pulse sequence described above. This is immediately followed with the acquisition of a second k-space image data set as indicated at process block 302 using the above-described unbalanced SSFP pulse sequence. The scanning parameters such as flip angle, TR, and echo time (TE) are kept the same from the first acquisition to the second acquisition. It is also important that the two image data sets are registered with each other and this may require the use of cardiac or respiratory gating to capture the subject in the same position for each acquisition. Navigator pulse sequences may also be used to correct for bulk subject motion between acquisitions.

As indicated at process block 304, images are then reconstructed from the two k-space image data sets. This is a complex two-dimensional Fourier transformation of each k-space data set to form corresponding complex images. Each complex image is then used to produce a corresponding magnitude image as indicated at process block 306. The magnitude of each pixel is calculated as the square root of the sum of the squares of the I and Q values of the pixel's complex value.

As indicated at process block 308, the final step is to subtract the two magnitude images. This is a pixel-by-pixel subtraction of the corresponding magnitude values in the balanced SSFP image and the unbalanced SSFP image. To completely remove the signal from background tissues the images may be weighted prior to performing this step. A weighting factor W_{fluid} may be used when relative contrast between arterial blood and synovial fluids is to be maximized, and a second weighting factor W_{edema} may be used when the relative contrast between arterial blood and edematous tissues is to be maximized. Also, an optimal weighting factor Wₒₚₜᵢₘₐₗ may be computed by averaging the weighting factors W_{fluid} and W_{edema}. The resulting magnitude difference image is an MR angiogram that brightly depicts the vasculature of interest while suppressing the surrounding, background tissues.

It should be apparent that many variations are possible from the above-described systems and methods. For example, the invention is applicable to 3D image acquisitions as well as 2D image acquisitions. One can use a T₂-weighted magnetization preparation sequence prior to each SSFP pulse sequence to suppress MR signals from veins. Other preparatory sequences such as presaturation and fat suppression may also be used.

Therefore, a system and method is provided to quickly acquire and produce an MR angiogram without the use of a contrast agent. It is a discovery of the present invention that the MR signal from moving spins in an unbalanced SSFP data acquisition is suppressed, whereas the MR signal from moving spins in a balanced SSFP data acquisition is bright. The MR signal from background tissues in both reconstructed magnitude images is substantially the same such that when they are subtracted, background tissue is substantially nulled and the signal from moving blood in the vasculature of interest is bright. Contrast is provided by spin motion without the use of contrast agents and without the time consuming addition of motion encoding gradients or preparatory pulse sequences.

The present invention is able to utilize the following pulse sequences that are available on commercially available MRI systems, for example, such as the following as well as others:

| Manufacturer | Balanced SSFP | Unbalanced SSFP |
|---|---|---|
| Siemens | True FISP | FISP |
| GE | FIESTA | MPGR, ORE |
| Philips | Balanced FFE | FFE |

The present invention has been described in terms of one or more preferred embodiments, and it should be appreciated that many equivalents, alternatives, variations, and modifications, aside from those expressly stated, are possible and within the scope of the invention.

1. A method for producing an angiogram of a subject with a magnetic resonance imaging (MRI) system without the use of a contrast agent, comprising the steps of:
a) acquiring, with the MRI system, a first image data set of vasculature of interest using a balanced steady-state free precession (SSFP) pulse sequence;
b) acquiring, with the MRI system, a second image data set of the vasculature of interest using an unbalanced SSFP pulse sequence;
c) reconstructing first and second images from the respective first and second acquired image data sets; and
d) subtracting a first of the first and second images from a second of the first and second images to produce an angiogram of the vasculature of interest.

2. The method of claim 1 wherein the balanced SSFP pulse sequence includes a slice select and readout gradient configured to have a nulled first moment that rephases signals from moving spins in the vasculature of interest between performances of the balanced SSFP pulse sequence.

3. The method of claim 1 wherein the unbalanced SSFP pulse sequence includes a slice select and readout gradient configured to dephase signals from moving spins in the vasculature of interest between performances of the unbalanced SSFP pulse sequence.

4. The method of claim 1 wherein signal corresponding to moving spins in the vasculature of interest appear dark in the angiogram.

5. The method of claim 1 wherein the balanced SSFP pulse sequence and the unbalanced SSFP pulse sequence share common scanning parameters including at least one of flip angle, repetition time (TR), and echo time (TE).

6. The method of claim 1 wherein step b) includes acquiring the second image data set such that the first image data set and the second image data set are registered.

7. The method of claim 1 wherein step b) includes performing at least one of cardiac gating and respiratory gating to register the first image data set and the second image data set.

8. The method of claim 1 wherein steps a) and b) include performing a navigator pulse sequences to correct for bulk of the subject steps a) and b).

9. The method of claim 1 wherein step c) includes performing a complex Fourier transformation of the first image data set and the second image data set to form corresponding complex images.

10. The method of claim 9 wherein step c) includes calculating a magnitude of each pixel as the square root of a sum of the squares of I and Q values of each pixel's complex value to produce first and second magnitude images.

11. The method of claim 10 wherein step d) includes performing a pixel-by-pixel subtraction of corresponding magnitude values in the first and second magnitude images.

12. The method of claim 10 wherein step d) includes weighting the first and second magnitude images prior to performing a subtraction of the first and second magnitude images to produce the angiogram of the vasculature of interest.

13. The method of claim 12 wherein a weighting factor W_{fluid} is used to adjust a relative contrast between arterial blood and synovial fluids.

14. The method of claim 12 wherein a weighting factor W_{edema} is used to adjust a relative contrast between arterial blood and edematous tissues.

15. The method of claim 12 wherein an optimizing weighting factor Wₒₚₜᵢₘₐₗ is used to adjust a first weighting factor W_{fluid}, which is used to adjust a relative contrast between arterial blood and synovial fluids, and a second weighting factor W_{edema}, which is used to adjust a relative contrast between arterial blood and edematous tissues.

16. The method of claim 1 wherein the balanced SSFP pulse sequence and the unbalanced SSFP pulse sequence are one of 3D pulse sequences and 2D pulse sequences.

17. The method of claim 1 further comprising performing a T₂-weighted magnetization preparation sequence prior steps a) and b) to suppress MR signals from veins.

18. The method of claim 1 further comprising performing at least one of a presaturation preparatory pulse sequence before steps a) and b) and a fat suppression technique.

19. The method of claim 1 wherein the vasculature of interest is substantially free of contrast agents.

20. The method of claim 1 wherein the first and second images are complex images and step d) includes performing a complex subtraction of the first of the first and second images from the second of the first and second images to produce the angiogram of the vasculature of interest
